(19) United States
(12) Patent Application Publication
(10) Pub. No.: US 2019/0240455 A1
(43) Pub. Date: Aug. 8, 2019

(54) CATHETER FEEDER

(71) Applicant: Bryan Tamao Archpru, San Jose, CA (US)

(72) Inventor: Bryan Tamao Archpru, San Jose, CA (US)

(21) Appl. No.: 16/246,432

(22) Filed: Jan. 11, 2019

Related U.S. Application Data
(60) Provisional application No. 62/626,351, filed on Feb. 5, 2018.

(51) Int. Cl. A61M 25/01; A61M 25/06

(52) U.S. Cl. CPC A61M 25/0113; A61M 25/0606; A61M 25/0612; A61M 25/065

(57) ABSTRACT

A catheter feeder has a connector for accepting engagement of a hollow needle. A top region, adjacent to the connector, has a first chamber sized to receive a catheter. The first chamber extends through the top region and through the connector allowing the catheter to enter the hollow needle through the first chamber. A handle has a second chamber sized to receive the catheter. The chamber tunnels completely through the handle. A curved thumb slide, between the top region and the handle, is composed of a region between the first chamber and the second chamber where the catheter is exposed and available to be manipulated by a thumb of an operator.

### BACKGROUND

Continuous peripheral nerve catheters (CPNCs) are used in the field of Anesthesia for the continuous delivery of local anesthesia. CPNCs are commonly placed in people to help reduce postoperative surgical pain and for other purposes. The use of CPNCs for regional delivery of local anesthesia is increasing because of the benefits to patient satisfaction and surgical recovery. The local anesthesia delivered by CPNCs can provide a continuous local “nerve block” to a patient experiencing discomfort or pain commonly associated with surgery. Throughout the following discussions, a CPNC is referred to as a catheter and the hollow continuous peripheral nerve block (CPNB) needle is referred to as the needle. Several techniques regarding the placement of these catheters exist. The following claims relate to the through-the-needle (TTN) catheter placement technique, which is a common technique used to place CPNCs.

To place a catheter in a patient, using the TTN technique, the hollow needle must first be inserted into a patient so that the tip of the needle is positioned in the desired location near the targeted nerve. The catheter is then threaded through the proximal end of the needle and advanced forward so that the tip of the catheter exits the distal end of the needle. The needle is then withdrawn while the catheter is advanced in such a way that the tip of the catheter continues to stay in the desired location near the nerve.

Withdrawing the needle so that the tip of the catheter continues to stay in the desired location requires both hands of the person performing the procedure. One hand is used to hold, stabilize, and retract the needle. The other hand handles and threads the catheter forward. While the hand holding the catheter advances the catheter through the needle, the needle is then withdrawn using the hand already holding the needle. Once the needle is completely out of the body, the rest of the catheter is threaded through the distal end of the needle tip and the needle is put away. The catheter can then be secured down to the patient's skin and local anesthetic infused through the proximal end of the catheter. The local anesthetic travels from the proximal end of the catheter, through the catheter, and is delivered near the desired nerve via the distal end of the catheter.

Ultrasonography, a technique that uses high frequency sound waves to visually differentiate various tissue densities within the body, is commonly used during the placement of the catheter. Ultrasonography allows the anesthesia provider to view and identify pertinent anatomical structures as well as monitor the insertion, advancement and final position of the needle. Using ultrasonography, the anesthesia provider who is placing the CPNC is able to visualize in real time the nerve, muscle, and vascular structures as the needle advances toward its intended target. This gives the anesthesia provider the ability to avoid contacting and injuring unwanted anatomy (i.e. blood vessels) with the needle.

During placement of the needle, the anesthesia provider controls the needle in one hand, while holding and scanning the ultrasound probe in the opposite hand. The ultrasound probe sends and receives the high frequency sound waves and is connected via a cable to a monitor, which displays real time ultrasonography images of the portion of the body being scanned. The anesthesia provider can use an ultrasound monitor to view and observe the location and progress of the needle in the patient in real time. The anesthesia provider commonly approaches the catheter placement by maintaining a long axis view of the needle once it has entered the patient's body. The anesthesia provider then attempts to keep the needle in view as the needle approaches the intended target. That is, one hand of the anesthesia provider is used to hold the ultrasound probe against the patient's body (viewing the desired anatomy and needle movement), while the other hand of the anesthesia provider is used to advance the needle toward its intended target under direct ultrasound visualization. Once the needle tip is in the desired location, local anesthetic or saline solution can be deployed to ensure correct needle tip placement and to open up a space near or around the nerve to accommodate the entry of the catheter.

Current common techniques used to place the CPNC via the TTN technique include the following examples. Once the needle tip is in the targeted location, the anesthesia provider can put down the ultrasound probe in order to allow the use of both hands. The anesthesia provider will use one hand to thread the catheter through the needle while the other hand stabilizes and then retracts the needle out of the patient's body. Because the ultrasound probe in put down, real time visualization of the catheter exiting the needle is temporarily lost. Furthermore, the anesthesia provider is unable to visualize the catheter tip location and movement while the needle is being withdrawn. Once the needle is out of the patient's body the anesthesia provider will commonly pick up and re-scan with the ultrasound probe to visualize the catheter or confirm catheter tip placement by visualizing, under ultrasound, the injection of local anesthetic, saline solution, or air in real time.

If continuous ultrasound monitoring is desired throughout this process, a second person can hold the ultrasound probe so that the anesthesia provider can use both hands to thread the catheter and retract the needle. This way, they can watch the catheter exit the distal tip of the needle in real time and advance it to its desired location under direct visualization. Once the catheter tip is in the desired place, the anesthesia provider can withdraw the needle while continuing to visually confirm that the catheter tip is maintained in the desired location. Using ultrasound monitoring while placing the catheter in real time allows the anesthesia provider to visualize the catheter exit from the needle tip and to watch how far beyond the tip the catheter extends. Using continuous ultrasound monitoring while withdrawing the needle may decrease the risk of the catheter tip inadvertently being pulled back too far with the retraction of the needle, resulting in mal-position of the catheter tip and a potentially failed nerve block.

### BRIEF DESCRIPTION OF THE DRAWINGS

FIG. 1 is a simplified diagram showing a catheter feeder ready to be attached to a regional block needle in accordance with an implementation.

FIG. 2 is a simplified diagram showing a catheter feeder attached to a regional block needle in accordance with an implementation.

FIG. 3 illustrates a process where a catheter feeder is used to place a catheter within a patient in accordance with an implementation.

FIG. 4 illustrates a catheter extending through chambers within a catheter feeder in accordance with an implementation.

FIG. 5 is a simplified diagram showing details of a catheter feeder in accordance with an implementation.

FIG. 6 is a simplified diagram showing a catheter feeder attached to a continuous peripheral nerve block needle ready to be used to place a catheter in a patient in accordance with an implementation.

### DESCRIPTION OF THE EMBODIMENTS

Continuous peripheral nerve catheters (CPNCs), herein referred to as catheters, are commonly placed within a patient to help with postoperative surgical pain. Such catheters may be inserted and placed within a patient using a through-the-needle (TTN) catheter placement technique. To assist placement of a catheter, a catheter feeder may be connected to and securely engaged to a hollow continuous peripheral nerve block (CPNB) needle, as described herein. These hollow CPNB needles commonly have a female luer lock connector fitted to the proximal end and often a Tuohy-type beveled tip. The catheter feeder securely engages into the proximal end of a CPNB needle and gives the anesthesia provider, who is the operator of the catheter feeder, the ability to control both needle movement (i.e. forward, backward, redirection, etc.) within the patient's body and catheter advancement and retraction with a single hand.

When the anesthesia provider has the ability to control both needle movement and catheter threading with a single hand, the other hand of the anesthesia provider is freed up to hold and control an ultrasound probe. This gives the anesthesia provider the ability to use their own two hands to continuously monitor, via ultrasonography, the movement of the needle throughout the procedure, while also visually confirming catheter tip placement even when the needle is being retracted from the patient's body. With the aid of the catheter feeder, the anesthesia provider can do this all under real time visualization without the help of a second person to hold the ultrasound probe.

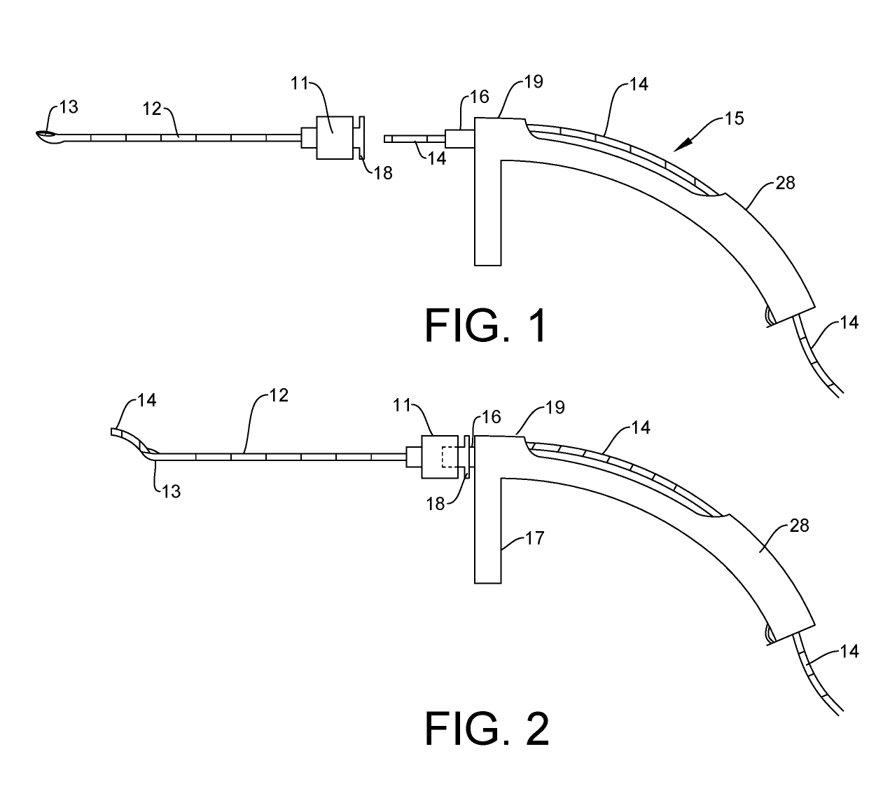

FIG. 1 shows a hollow continuous peripheral nerve block (CPNB) needle 12 having a Tuohy-type, anti-coring curved shape at the distal tip 13 of needle 12. The anti-coring curved shape at distal tip 13 of needle 12 is designed to cause a catheter to exit distal tip 13 at an angle. A fitting 11 at a proximal end of the needle 12 includes a female luer lock connector 18. A catheter feeder 10 includes a male luer slip connector 16 that receives a female luer lock connector 18 to hold needle 12 snugly to catheter feeder 10. Once this secure connection is made, the catheter feeder and CPNB needle can be advanced, retracted, and redirected together by simply holding onto the catheter feeder alone or a hand grip that holds both the catheter feeder and needle. A continuous peripheral nerve catheter 14 is fed through a chamber in a handle 28 of catheter feeder 10, through a catheter exposed region 15 and into a chamber in a top region 19 then through hollow male luer slip connector 16 into needle 12. The curved shape of catheter feeder 10 helps avoid kinking of catheter 14 as it is fed through catheter feeder 10. The curved shape of catheter feeder 10 forces catheter 14 to have a curved shape as it passes through catheter exposed region 15. A trigger 17 of catheter feeder 10 allows for a comfortable resting place of the index finger of an anesthesia provider and also allows for counter traction as the anesthesia provider uses a thumb to apply pressure to catheter 14 at exposed region 15 to feed and retract catheter 14 through catheter feeder 10 into needle 12. Exposed region 15 thus acts as a thumb slide for feeding and retracting the catheter 14. The anesthesia provider uses catheter feeder 10 to control feeding and retracting of catheter 14 though needle 12 when precisely locating the tip of catheter 12 within a patient, and uses catheter feeder 10 to continue feeding catheter 14 though catheter feeder 10 as needle 12 is withdrawn from the patient after the tip of catheter 12 is precisely located within the patient.

Female luer lock connector 18 can receive both a male luer lock connector and a male luer slip connector. Male luer slip connector 16 is engaged to female luer lock connector 18 by firmly inserting and applying a slight twisting motion. This enables adequate engagement and stability between catheter feeder 10 and needle 12 so that needle 12 can be advanced into or withdrawn from the patient by holding onto and advancing or withdrawing catheter feeder 10. This type of connection also allows for easy disconnection by applying a similar twisting motion and pulling needle 12 and catheter feeder 10 in opposite directions.

FIG. 2 shows needle 12 assembled to catheter feeder 10 where female luer lock connector 18 at the proximal end of needle 12 has been firmly fixed in position over male luer slip connector 16 of catheter feeder 10. Catheter 14 has been fed through needle 12, for example by an anesthesia provider using the thumb to apply pressure to catheter 14 at exposed region 15 to feed catheter 14 through into and through needle 12 so that catheter 14 extends out of needle 12 at distal tip 13.

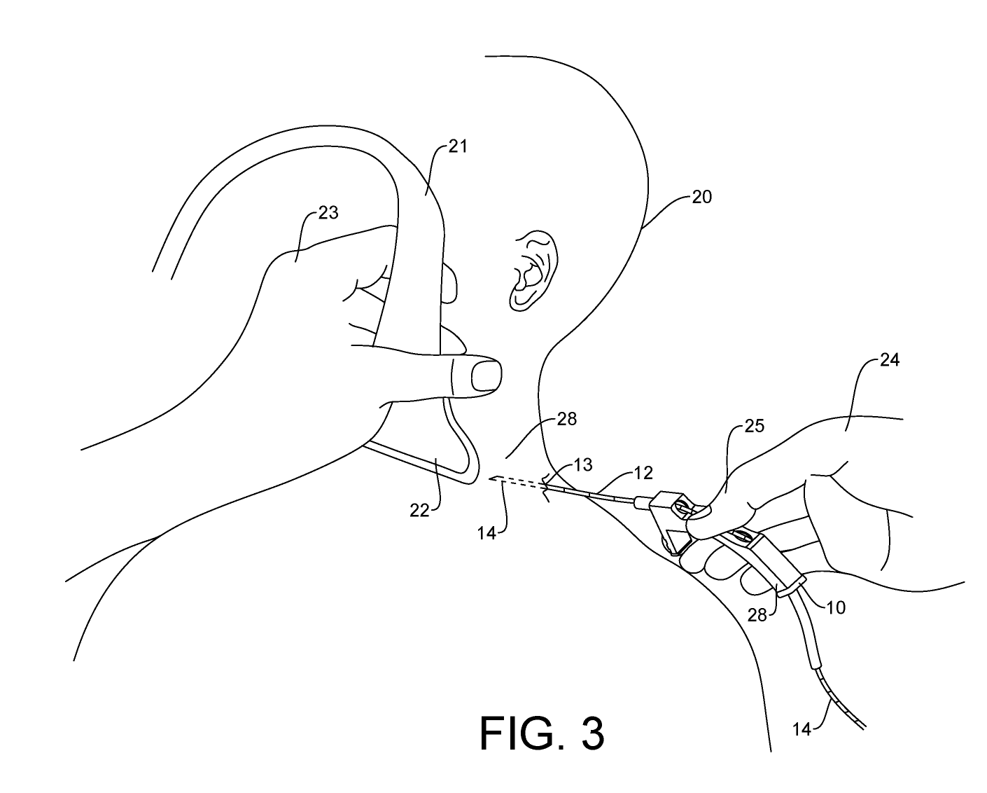

FIG. 3 shows an anesthesia provider using needle 12 and catheter feeder 10 to place catheter 14 within a patient 20. With a thumb 25 of a hand 24, the anesthesia provider applies pressure to catheter 14 at exposed region 15 to feed and retract catheter 14 through catheter feeder 10 and through needle 12 so as to precisely locate the tip of catheter 12 within patient 20. Once correct placement is achieved, the anesthesia provider uses thumb 25 of hand 24, to continue feeding catheter 14 though catheter feeder 10 as the anesthesia provider withdraws needle 12 from patient 20, while leaving the tip of catheter 12 precisely located within patient 20 at a desired location 28. The other hand 23 of the anesthesia provider is used to hold the transducer end 22 of an ultrasound probe 21 against the patient so as to monitor the process of precise placement of the tip of catheter 12 within patient 20 and withdrawal of needle 12 from patient.

Catheter feeder 10 can be attached to needle 12 before skin puncture, so that the tip of catheter 14 is already threaded through catheter feeder 10 and needle 12 and so that the tip of catheter 14 is in position near distal tip 13 of needle 12. Alternatively, catheter feeder 10 can be attached to needle 12 after needle and needle tip 12 is already confirmed to be in the desired position within the body of patient 20. During the procedure, once the distal tip 13 is in a desired position, catheter feeder 10 is engaged, and catheter 14 is threaded up to distal tip 13 of needle 12. Thumb 25 of hand 24 will push the catheter 14 forward using the thumb slide at exposed region 15. Hand 23 controls ultrasound probe 21 so that the anesthesia provider can view needle 12 in long axis, and so that the anesthesia provider can view catheter 14 exiting distal tip 13 of needle 12. The anesthesia provider will continue to manipulate catheter 14 using thumb 25 of hand 24 at exposed region 15 until the catheter tip is in satisfactory position. The anesthesia provider can then pull back on trigger 17 and curved handle 28 of catheter feeder 10 to withdraw needle 12 while simultaneously threading catheter 14 forward using thumb 25 of hand 24 at exposed region 15. This can be continued until the needle 12 tip has exited the skin of patient 20. During the removal of needle 12, the tip of catheter 14 remains in the desired position within patient 20. Once entire needle 12 is removed from patient 20, the remainder of catheter 20 can be passed through distal tip 13 of needle 12 and needle 12 and catheter feeder 10 can be put away. The catheter 14 can then be secured to patient 20 and local anesthesia administered through catheter 14 in accordance with customary practice.

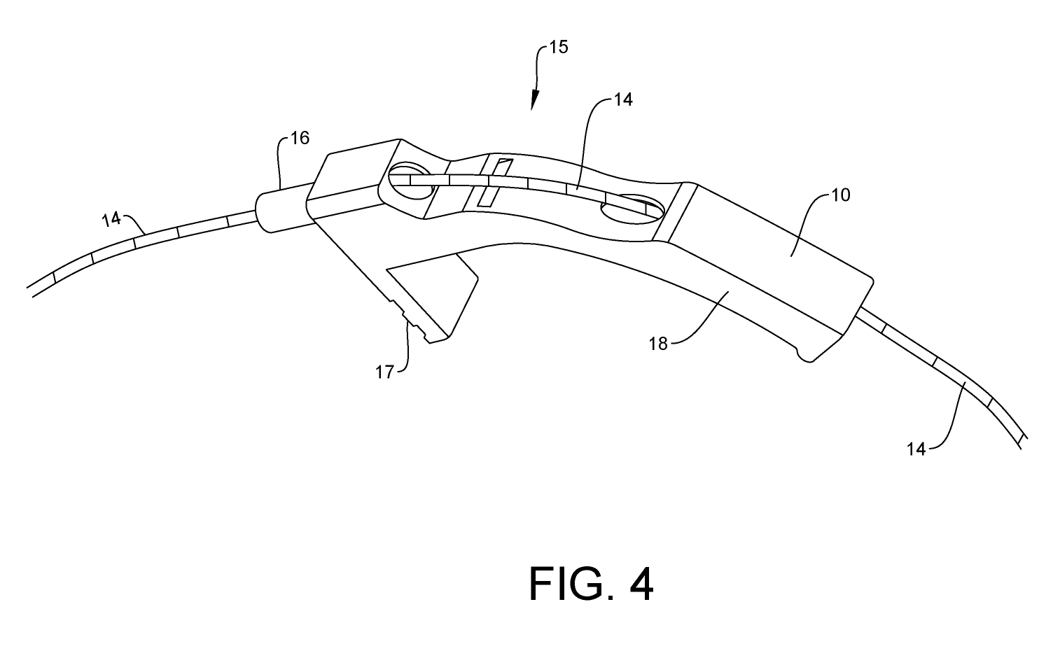

FIG. 4 illustrates a path of catheter 14 as catheter 14 is fed through a chamber in handle 28 of catheter feeder 10, through catheter exposed region 15 and through a chamber in a top region 19.

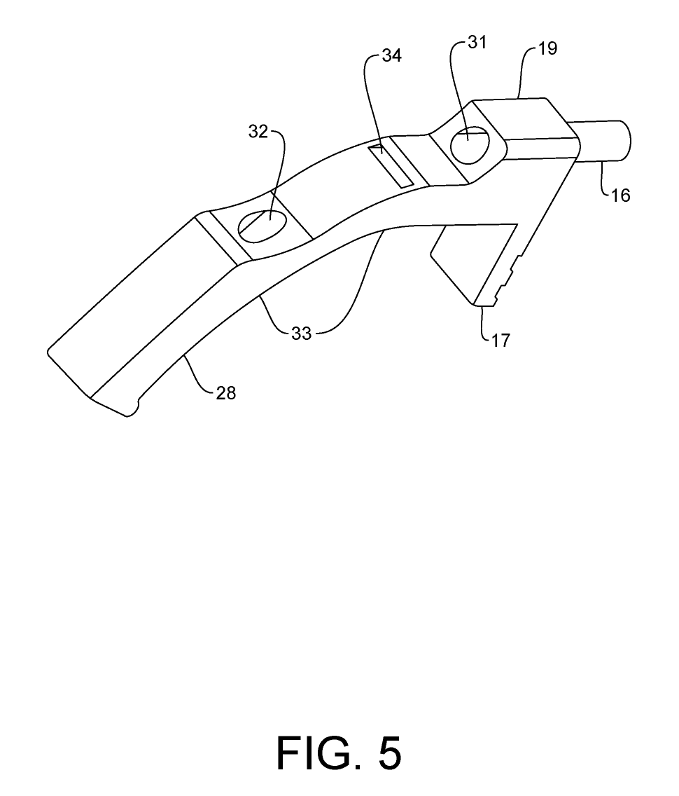

FIG. 5 shows location of a chamber 31 in top region 19 and a location of a chamber 32 in handle 28 of catheter feeder 10. An indented ridge 34 is added in exposed region 15 to provide traction between the thumb of the anesthesia provider and catheter 14 and to aid in implementing the thumb slide. The curved shape of body 33 of catheter feeder 10 helps prevent catheter 14 from kinking while providing a surface area that allows the anesthesia provider to apply counter pressure to the pressure exerted by the thumb. The curvature between chamber 31 and chamber 32 forms a region between chamber 31 and chamber 32, which serves as a thumb slide, where the catheter is exposed and available to be manipulated by a thumb of an operator such as the anesthesia provider. The curved shape also provides a place for leverage so that once the catheter tip is precisely located within the patient, the anesthesia provider can easily pull on the curved shape of catheter feeder 10 to remove needle 12 from the patient while the thumb of the anesthesia provider controls continued feeding of catheter 14 through needle 12 so that the catheter tip remains precisely placed within the patient while needle 12 is withdrawn.

Once catheter feeder 10 has catheter 14 threaded through chamber 32 and chamber 31, catheter feeder 10 can be engaged into the proximal end of needle 12 by firmly pressing catheter feeder 10 against needle 12 and applying a twisting motion. The male luer slip type design of catheter feeder 10 enables it to be engaged and fitted tightly into the standard proximal hub of needle 12. The engagement is secure enough so that both catheter feeder 10 and needle 12 can be advanced and withdrawn together as one combined tool, simply by holding onto catheter feeder 10 or using a hand grip in a way that holds both the proximal end of needle 12 and catheter feeder 10. The thumb of the hand holding catheter feeder 10 applies downward pressure on catheter 14 at exposed regions 15 to stabilize catheter feeder 10 and catheter 14 while manipulating needle 12 within a patient.

Trigger 17 and curved handle 28 on catheter feeder 10 can comfortably accommodate the remaining fingers of hand 24 in varying positions that the anesthesia provider chooses. These fingers help to grip catheter feeder 10 and apply counter pressure to the thumb, stabilizing catheter feeder 10 when in motion. Catheter feeder 10 can also advance or withdraw the catheter 14 at any time during the procedure, by pushing the catheter 14 forward or pulling it back along the exposed region 15 under thumb 25. By having independent control of both needle 12 movement and the movement of catheter 14 using a single hand, this frees the anesthesia provider's other hand to hold and control an ultrasound probe. This allows the anesthesia provider to both control needle and catheter movement and monitor the procedure without aid from a second person.

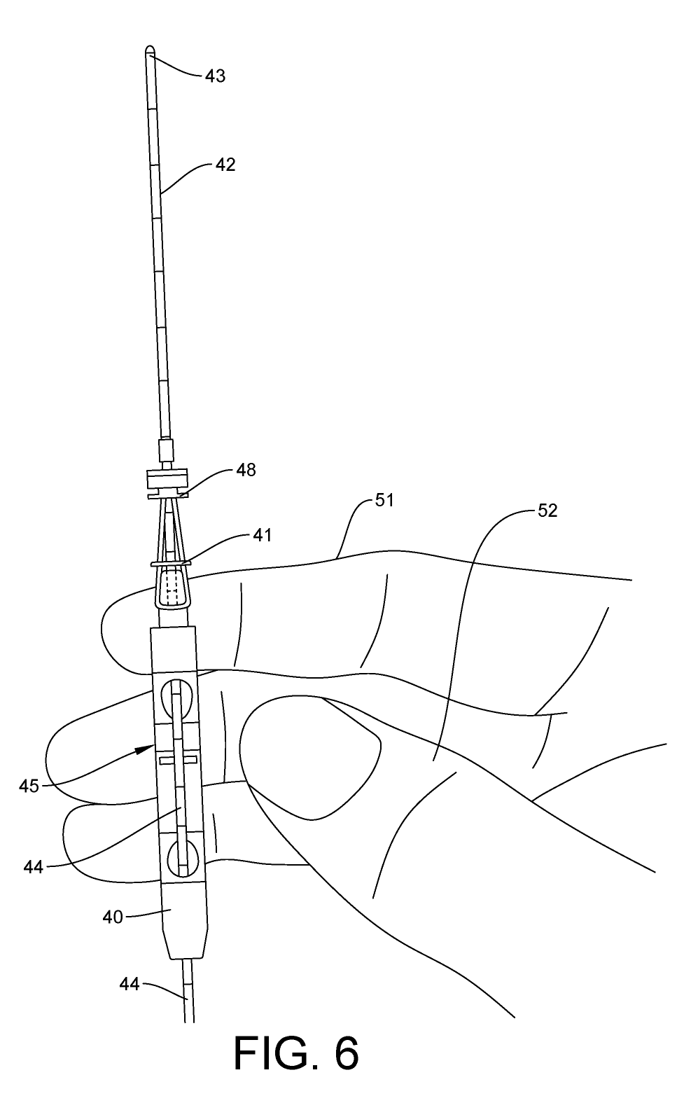

FIG. 6 illustrates that the style and shape of the catheter feeder and the needle can be varied. FIG. 6 shows a hollow continuous peripheral nerve block (CPNB) needle 42 having an anti-coring curved tip 43 designed to cause a catheter exit the needle tip at an angle. A fitting 41 at a proximal end of the needle 42 includes a female luer lock connector 48. A catheter feeder 40 includes a male luer slip connector that receives female luer lock connector 48 to hold needle 42 snugly to catheter feeder 40. A continuous peripheral nerve catheter 44 is fed through catheter feeder 40, through a catheter exposed region 45. The thumb 52 of a hand 51 of the anesthesia provider controls feeding and retracting of catheter 44 though needle 42 when precisely locating the tip of catheter 42 within a patient, and when needle 42 is withdrawn from the patient after the tip of catheter 42 is precisely located within the patient.

Use of catheter feeder 10 may lead to increased accuracy of catheter tip placement, decreased accidental dislodgement of the catheter while withdrawing the regional block needle, faster average speed of placing these nerve catheters and elimination of the need for a second person to hold an ultrasound probe.

The foregoing discussion discloses and describes merely exemplary methods and embodiments. As will be understood by those familiar with the art, the disclosed subject matter may be embodied in other specific forms without departing from the spirit or characteristics thereof. Accordingly, the present disclosure is intended to be illustrative, but not limiting, of the scope of the invention, which is set forth in the following claims.

What is claimed is:

1. A catheter feeder, comprising:
a connector for accepting engagement of a hollow needle;
a top region adjacent to the connector, the top region having a first chamber sized to receive a catheter, the first chamber extending through the top region and through the connector allowing the catheter to enter the hollow needle through the first chamber;
a handle, the handle having a second chamber sized to receive the catheter, the chamber tunneling completely through the handle; and,
a curved thumb slide between the top region and the handle, the curved thumb slide composed of a region between the first chamber and the second chamber where the catheter is exposed and available to be manipulated by a thumb of an operator, the curved thumb slide forcing the catheter to have a curved shape where the catheter passes through the region between the first chamber and the second chamber.

2. A catheter feeder as in claim 1 where the connector is a male luer slip connector.

3. A catheter feeder as in claim 1 wherein the hollow needle is a hollow continuous peripheral nerve block needle.

4. A catheter feeder as in claim 1, additionally comprising:
a trigger adjacent to the top region, the trigger extending in a direction approximately perpendicular to the first chamber, the trigger providing support to fingers of the operator.

5. A catheter feeder as in claim 4, wherein the curved thumb slide is curved so that the catheter exits the second chamber and curves along the curved thumb slide before entering the first chamber.

6. A catheter feeder as in claim 1, wherein the curved thumb slide is curved so that the catheter exits the second chamber and curves along the curved thumb slide before entering the first chamber.

7. A catheter feeder as in claim 1 wherein the hollow needle is a hollow continuous peripheral nerve block needle with a female luer lock connector.

8. A catheter feeder as in claim 1 wherein the catheter is a continuous peripheral nerve catheter.

9. A system configured for the continuous delivery of local anesthesia, comprising:
a catheter, the catheter being a continuous peripheral nerve catheter;
a hollow needle, the hollow needle being a hollow continuous peripheral nerve block needle;
a catheter feeder, comprising:
a connector for accepting engagement of the hollow needle,
a top region adjacent to the connector, the top region having a first chamber sized to receive the catheter, the first chamber extending through the top region and through the connector allowing the catheter to enter the hollow needle through the first chamber,
a handle, the handle having a second chamber sized to receive the catheter, the chamber tunneling completely through the handle, and
a curved thumb slide between the top region and the handle, the curved thumb slide composed of a region between the first chamber and the second chamber where the catheter is exposed and available to be manipulated by a thumb of an operator, the curved thumb slide forcing the catheter to have a curved shape where the catheter passes through the region between the first chamber and the second chamber.

10. System as in claim 9 where the connector is a male luer slip connector.

11. System as in claim 9, additionally comprising:
a trigger adjacent to the top region, the trigger extending in a direction approximately perpendicular to the first chamber, the trigger providing support to fingers of the operator.

12. System as in claim 11, wherein the curved thumb slide is curved so that the catheter exits the second chamber and curves along the curved thumb slide before entering the first chamber.

13. System as in claim 9, wherein the curved thumb slide is curved so that the catheter exits the second chamber and curves along the curved thumb slide before entering the first chamber.

14. System as in claim 9 wherein the hollow needle is a hollow continuous peripheral nerve block needle with a female luer lock connector.

15. A catheter feeder, comprising:
a connector for accepting engagement of a hollow needle;
a top region adjacent to the connector, the top region having a first chamber sized to receive a catheter, the first chamber extending through the top region and through the connector allowing the catheter to enter the hollow needle through the first chamber;
a handle, the handle having a second chamber sized to receive the catheter, the chamber tunneling completely through the handle; and,
a curved thumb slide between the top region and the handle, the curved thumb slide composed of a curved region between the first chamber and the second chamber where the catheter is exposed and available to be manipulated by a thumb of an operator, wherein the curved region is curved so that the catheter exits the second chamber and curves before entering the first chamber, the curved region forcing the catheter to have a curved shape where the catheter passes through the curved region.

16. A catheter feeder as in claim 15 where the connector is a male luer slip connector.

17. A catheter feeder as in claim 15 wherein the hollow needle is a hollow continuous peripheral nerve block needle.

18. A catheter feeder as in claim 15, additionally comprising:
a trigger adjacent to the top region, the trigger extending in a direction approximately perpendicular to the first chamber, the trigger providing support to fingers of the operator.

19. A catheter feeder as in claim 15 wherein the hollow needle is a hollow continuous peripheral nerve block needle with a female luer lock connector.

20. A catheter feeder as in claim 15 wherein the catheter is a continuous peripheral nerve catheter.
